(19) United States
(12) Patent Application Publication
(10) Pub. No.: US 2012/0121510 A1
(43) Pub. Date: May 17, 2012

(54) LOCALIZED THERAPY FOLLOWING BREAST CANCER SURGERY

(76) Inventors: Rachel F. Brem, Owings Mills, MD (US); Daniel S. Kohane, Newton, MA (US); Robert S. Langer, Newton, MA (US)

(21) Appl. No.: 13/294,346

(22) Filed: Nov. 11, 2011

Related U.S. Application Data
(60) Provisional application No. 61/412,602, filed on Nov. 11, 2010.

(51) Int. Cl. A61K 49/00; A61K 31/175; A61P 17/02; A61K 31/704; A61P 35/00; A61K 33/24; A61K 31/337

(52) U.S. Cl. 424/9.1; 424/649; 514/589; 514/449; 514/34

(57) ABSTRACT

Particles providing prolonged release of chemotherapy are injected or implanted into surgical sites in the breast following removal of cancerous tissue. In one embodiment, the particles are designed to not release formulation for approximately two to three weeks after surgery so as to not inhibit healing; in another embodiment particles are not administered until two to three weeks after surgery, and release immediately. The particles then release an effective amount of a chemotherapeutic such as a taxane to inhibit proliferation of any remaining cancer cells at or near the surgical site. This may also help prevent overproliferation leading to scarring with the surgical region.

### CROSS REFERENCE TO RELATED APPLICATIONS

This application claims benefit of and priority to U.S. Ser. No. 61/412,602 filed Nov. 11, 2010, the contents of which are incorporated herein by reference in its entirety.

### FIELD OF THE INVENTION

The present invention is generally in the field localized treatment following surgery for breast cancer to prevent reoccurrence.

### BACKGROUND OF THE INVENTION

Breast cancer is an insidious disease affecting thousands of women daily.

Women with breast cancer have many treatment options. The treatment that's best for one woman may not be best for another. The treatment options are surgery, radiation therapy, hormone therapy, chemotherapy, targeted therapy, and combinations thereof. Surgery and radiation therapy are types of local therapy. They remove or destroy cancer in the breast. Hormone therapy, chemotherapy, and targeted therapy are types of systemic therapy. The drug enters the bloodstream and destroys or controls cancer throughout the body.

The treatment that is used depends mainly on the stage of the cancer, the results of the hormone receptor tests, the result of the HER2/neu test, the results of specifics parameters of the tumor itself and the general health.

Surgery is the most common treatment for breast cancer. Breast-sparing surgery is an operation to remove the cancer but not the breast. It can be a lumpectomy or a segmental mastectomy (also called a partial mastectomy). Sometimes an excisional biopsy is the only surgery a woman needs because the surgeon removed the whole lump with margins which are negative for cancer. Mastectomy is an operation to remove the entire breast (or as much of the breast tissue as possible). In some cases, a skin-sparing mastectomy may be an option. For this approach, the surgeon removes as little skin as possible. The surgeon usually removes one or more lymph nodes from under the arm to check for cancer cells. If cancer cells are found in the lymph nodes, other cancer treatments will be needed. In almost all cases of surgery which is not mastectomy, surgery is followed by radiation.

If there is systemic involvement (i.e., cancer cells are found in the lymph nodes), the patient is may be treated with surgery and with chemotherapy, hormonal therapy, and/or targeted therapy, as well as surgery.

After successful treatment, patients are monitored long term to insure that the cancer does not return. Usually the physician will conduct a physical exam and either a mammogram and/or an MRI, a PET scan or other imaging. If the examination is negative, the patient is told to return again in three to six months. If the patient is now positive for cancer, the cancer is re-treated. The prognosis is usually not as favorable when there is a recurrence of the cancer.

It is an object of the present invention to decrease the likelihood of cancer reoccurring.

### SUMMARY OF THE INVENTION

Particles providing prolonged release of chemotherapy are injected or implanted into surgical sites in the breast following removal of cancerous tissue. In one embodiment, the particles are designed to not release formulation for approximately two to three weeks after surgery so as to not inhibit healing; in another embodiment particles are not administered until two to three weeks after surgery, and release immediately. The particles then release an effective amount of a chemotherapeutic such as a taxane to inhibit proliferation of any remaining cancer cells at or near the surgical site. This may also help prevent overproliferation leading to scarring with the surgical region. In a preferred embodiment, release occurs for six to twelve months. Upon a negative re-examination, the particles are re-injected into the surgical site to prevent reoccurrence. In a particularly preferred embodiment the particles also contain an imaging agent so that they can be imaged and/or removed if necessary. The process of application can be repeated as often and as frequently as needed to prevent recurrence of disease.

### DETAILED DESCRIPTION OF THE INVENTION

### I. Formulations

Formulations consist of particles which can be suspended in a suitable carrier or applied as a dry powder to a surgical site. The particles are preferably formed of a natural or synthetic polymer, and may be degradable or non-degradable. The particles contain chemotherapeutic which is released in an effective amount to prevent proliferation of any residual cancer cells with the region where the particles are injected, over a prolonged period of time, six to twelve months, after administration to the site where release is to occur. The particles also contain an imaging agent, preferably an agent detectable in a mammogram, by ultrasound, by x-ray, or by MRI.

### A. DEFINITIONS

“Contacting” means an instance of exposure by close physical contact of at least one substance to another substance.

“Admixture,” “mixture,” or “blend” is generally used herein to refer to a physical combination of two or more different components. In the case of polymers, an admixture, mixture, or blend of polymers is a physical blend or combination of two or more different polymers. The mixture may be homogeneous or heterogeneous.

“Chemotherapeutic” is a substance used for the treatment (e.g., therapeutic agent) and/or prevention (e.g., prophylactic agent) of the proliferation of cancer cells, either as a cytostatic (inhibiting proliferation) or as a cytotoxic (killing the cells). The chemotherapeutic may be active or function as a pro-drug, which becomes biologically active or more active after it has been placed in a predetermined physiological environment. Examples can include, but are not limited to, small-molecule drugs, peptides, proteins, antibodies, sugars, polysaccharides, nucleotides, oligonucleotides, aptamers, siRNA, nucleic acids, and combinations thereof.

“Sufficient” or “effective” mean an amount and/or time needed to achieve one or more desired result.

As used herein, a “wt. %” or “weight percent” or “percent by weight” of a component, unless specifically stated to the contrary, refers to the ratio of the weight of the component to the total weight of the composition in which the component is included, expressed as a percentage.

As used herein, a “mole percent” or “mole %” of a component, unless specifically stated to the contrary, refers to the ratio of the number of moles of the component to the total number of moles of the composition in which the component is included, expressed as a percentage.

“Biocompatible” as used herein refers to a material and any metabolites or degradation products thereof that are generally non-toxic to the recipient and do not cause any significant adverse effects to the subject based on criteria used by regulatory agencies such as the U.S. Food and Drug Administration.

“Biodegradable” refers to a material that will degrade or erode under physiologic conditions to smaller units or chemical species that are capable of being metabolized, eliminated, or excreted by the subject.

“Molecular weight” as used herein, unless otherwise specified, refers to the relative average chain length of the bulk polymer. In practice, molecular weight can be estimated or characterized in various ways including gel permeation chromatography (GPC) or capillary viscometry. GPC molecular weights are reported as the weight-average molecular weight (M_{w}) as opposed to the number-average molecular weight (Mₙ). Capillary viscometry provides estimates of molecular weight as the inherent viscosity determined from a dilute polymer solution using a particular set of concentration, temperature, and solvent conditions.

“Mean particle size” refers to the statistical mean particle size (diameter) of the particles in the composition.

“Copolymer” is used herein to refer to a single polymeric material that is comprised of two or more different monomers. The copolymer can be of any form, such as random, block, graft, etc. The copolymers can have any end-group, including capped or acid end groups.

“Controlled release” or “modified release”, as used herein, refers to a release profile in which the drug release characteristics of time course and/or location are chosen to accomplish therapeutic or convenience objectives not offered by conventional dosage forms such as solutions, suspensions, or promptly dissolving dosage forms. Delayed release, extended release, and pulsatile release and their combinations are examples of modified release.

“Delayed release” as used herein refers to release of a drug (or drugs) at a time other than promptly after administration.

“Extended release” as used herein refers to release of a drug (or drugs) that allows at least a twofold reduction in dosing frequency as compared to the drug presented as a conventional dosage form (e.g. as a solution or prompt drug-releasing, conventional solid dosage form).

“Pulsatile release” as used herein refers to release of a drug (or drugs) that mimics a multiple dosing profile without repeated dosing and allows at least a twofold reduction in dosing frequency as compared to the drug presented as a conventional dosage form (e.g. as a solution or prompt drug-releasing, conventional solid dosage form). A pulsatile release profile is characterized by a time period of no release (lag time) or reduced release followed by rapid drug release.

“Excipient” is used herein to include any other compound that can be contained in or on the particle that is not a therapeutic, prophylactic or diagnostic compound. As such, an excipient should be pharmaceutically or biologically acceptable or relevant, for example, an excipient should generally be non-toxic to the subject. “Excipient” includes a single such compound and is also intended to include a plurality of compounds.

The term “microparticle” is used herein to refer to structures or particles having sizes from about 10 nm to about 1000 microns and includes microcapsules, microspheres, nanoparticles, nanocapsules, nanospheres, as well as particles, in general that are less than about 1000 microns. The particles may be spherical or non-spherical in shape. A microcapsule or nanocapsule is generally a particle that has a heterogeneous structure whereby the particle is covered by a substance or coating of some type, often a polymer or polymeric material or a wall-forming material. When the particle contains an agent (such as a chemotherapeutic or diagnostic agent or other excipient or additive), the agent is generally heterogeneously distributed in the particle and is typically centrally located within the membrane or coating. A microcapsule can also include microbubbles (hollow particle), porous microbubbles, porous microcapsules, and particles in general that comprise a central core surrounded by a unique outer membrane. In contrast, a microsphere or nanosphere has a more homogeneous structure whereby any incorporated agents are more or less distributed throughout the matrix of the particle where the remainder of the matrix is comprised of a polymer or polymeric material or matrix-forming material. A microsphere or nanosphere can include porous microspheres or nanospheres.

As used herein, “solids content” refers to the weight percent of particles suspended in a liquid vehicle, calculated as weight of the particles divided by weight of the particles and vehicle.

“Needle” is used herein to refer to devices that can be used to administer, deliver, inject, or otherwise introduce a microparticle formulation to a subject for any purpose(s) including medical, clinical, surgical, therapeutic, pharmaceutical, pharmacological, diagnostic, cosmetic, and prophylactic purposes. Thus, as defined herein, needle includes needle, all needle-like devices, and all other annular microparticle introduction devices, such as tubing, etc. Specific examples include needles, hypodermic needles, surgical needles, infusion needles, catheters, trocars, cannulas, tubes, and tubing used for clinical, surgical, medical, procedural, or medical purposes.

“Injected”, “injection”, or “injectability” as used herein is intended to include any administration of the microparticle, such as by injection, infusion, or any other delivery through any annular delivery device to the subject. Injection includes delivery through a tube.

### B. Polymers

Particle compositions containing an active agent can be used as an injectable, long-acting (or modified release) drug-delivery formulation. Injectable particle compositions designed for drug-delivery purposes can release their active agent at a variety of different rates including release at a constant rate (zero-order rate of release), at a nearly constant rate (near zero-order or pseudo-zero-order), at a declining rate (first-order) and combinations thereof. The polymers or matrix-forming materials used to prepare the particles, and from which the drugs are released, must be biocompatible and also compatible with the drug. The polymer or matrix forming material can be biodegradable or absorbable by the subject. These materials must also be able to control the rate of release of the agent to be delivered, prevent any undesirable release (for example, rapid elution of a drug).

Polymers, copolymers, or combination (blends or admixtures) of polymers are used to prepare particles. Selection of the components, molecular weight, crystallinity, relative concentrations of components, copolymerization versus mixing, and addition of agents such as pore forming agents, can be used to manipulate degradation and therefore drug release.

The particles can be formed from homopolymers, copolymers or blends containing two or more polymers, such as homopolymers or copolymers or combinations thereof.

Surface eroding polymers such as polyanhydrides may be used to form the particles. For example, polyanhydrides such as poly[(p-carboxyphenoxy)-hexane anhydride] (PCPH) may be used. Biodegradable polyanhydrides are described in U.S. Pat. No. 4,857,311. In another embodiment, bulk eroding polymers such as those based on polyesters including poly(hydroxy acids) can be used. For example, polyglycolic acid (PGA), polylactic acid (PLA), or copolymers thereof may be used to form the particles. The polyester may also have a charged or functionalizable group, such as an amino acid. In a preferred embodiment, particles with controlled release properties can be formed of poly(D,L-lactic acid) and/or poly(D,L-lactic-co-glycolic acid) (“PLGA”) which incorporate a surfactant such as DPPC.

Other polymers include polyamides, polycarbonates, polyalkylenes such as polyethylene, polypropylene, poly(ethylene glycol), poly(ethylene oxide), poly(ethylene terephthalate), poly vinyl compounds such as polyvinyl alcohols, polyvinyl ethers, and polyvinyl esters, polymers of acrylic and methacrylic acids, celluloses and other polysaccharides, and peptides or proteins, or copolymers or blends thereof. Polymers may be selected with or modified to have the appropriate stability and degradation rates in vivo for different controlled drug delivery applications.

Polyester as used herein refers to polymers that contain the monomer unit:

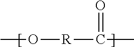

wherein R is a linear or branched, aliphatic or aromatic group. These include poly hydroxy acids such as poly(lactide), poly(glycolide), and copolymers thereof, polyhydroxyalkanoates such as poly(4-hydroxybutyrate) and poly(4-hydroxybutyrate-co-3-hydroxybutyrate), and polyanhydrides. These may be used as homopolymers, copolymers, blends, and mixtures. Other biocompatible or biodegradable polymers that can be blended or co-polymerized with the polymers described above include, but are not limited to, polycaprolactones; poly(orthoesters); poly(phosphazenes); synthetically or biologically prepared polyesters; poly(lactide-co-caprolactones); polycarbonates; tyrosine polycarbonates; polyamides (including synthetic and natural polyamides, polypeptides, and poly(amino acids)); polyesteramides; polyesters; poly(dioxanones); poly(alkylene alkylates); polyethers (such as polyethylene glycol, PEG, and polyethylene oxide, PEO); polyvinyl pyrrolidones or PVP; polyurethanes; polyetheresters; polyacetals; polycyanoacrylates; poly(oxyethylene)/poly(oxypropylene) copolymers; polyacetals, polyketals; polyphosphates; (phosphorous-containing) polymers; polyphosphoesters; polyhydroxyvalerates; polyalkylene oxalates; polyalkylene succinates; poly(maleic acids); chitin; chitosan; modified chitosan; biocompatible polysaccharides derivatized polysaccharides (jointly referred to herein as polysaccharides); and combinations thereof (including blends and block, random, or graft copolymers thereof). In one embodiment, copolymers that can be used include block copolymers containing blocks of hydrophilic or water soluble polymers, such as polyethylene glycol, (PEG) or polyvinyl pyrrolidone (PVP), with blocks of other biocompatible or biodegradable polymers, for example, poly(lactide), poly(lactide-co-glycolide, or polycaprolactone or combinations thereof.

Suitable biocompatible, non-biodegradable polymers include, but are not limited to, polyacrylates; ethylene-vinyl acetates; acyl substituted cellulose acetates; non-degradable polyurethanes; polystyrenes; polyvinyl chlorides; polyvinyl fluorides; poly(vinyl imidazoles); chlorosulphonate polyolefins; polyethylene oxides; or blend or copolymers thereof.

In one embodiment, the polymer is a biodegradable polyester containing monomers such as glycolide and/or lactide including polyglycolide, polylactide, and poly(lactide-co-glycolide) or mixtures thereof. These polymers are available with or without carboxylic acid end groups. When the end group of the poly(lactide-co-glycolide), poly(lactide), or polyglycolide is not a carboxylic acid, for example, an ester, then the resultant polymer is referred to herein as blocked or capped. The unblocked polymer, conversely, has a terminal carboxylic group (herein referred to as having an acid end-group). In one embodiment, linear lactide/glycolide polymers are used; however star polymers can be used as well. Molecular weight and crystallinity can be used to control rate of degradation, and release. For example, the lactide portion of the polymer has an asymmetric carbon. Racemic DL-, L-, and D-polymers are commercially available. The L-polymers are more crystalline and resorb slower than DL-polymers. In addition to copolymers containing glycolide and DL-lactide or L-lactide, copolymers of L-lactide and DL-lactide are also available.

Polymer blends can also be prepared from two or more particle formulations. In another aspect, particle compositions that differ in particle size can be blended together. The particle compositions that are blended can be similar in composition except for particle size or they can differ in composition and particle size. Particle compositions that are blended together can differ in a variety of ways, for example, polymer molecular weight, inherent viscosity, polymer composition (copolymer composition or the composition of a polymer admixture), choice of active agent, concentration of active agent, choice of excipient, concentration of excipient, distribution of agent in the particle, or any combination thereof.

Surfactants may be anionic, cationic, amphoteric or nonionic surface active agents. Suitable anionic surfactants include, but are not limited to, those containing carboxylate, sulfonate and sulfate ions. Examples of anionic surfactants include sodium, potassium, ammonium of long chain alkyl sulfonates and alkyl aryl sulfonates such as sodium dodecylbenzene sulfonate; dialkyl sodium sulfosuccinates, such as sodium dodecylbenzene sulfonate; dialkyl sodium sulfosuccinates, such as sodium bis-(2-ethylthioxyl)-sulfosuccinate; and alkyl sulfates such as sodium lauryl sulfate. Cationic surfactants include, but are not limited to, quaternary ammonium compounds such as benzalkonium chloride, benzethonium chloride, cetrimonium bromide, stearyl dimethylbenzyl ammonium chloride, polyoxyethylene and coconut amine. Examples of nonionic surfactants include ethylene glycol monostearate, propylene glycol myristate, glyceryl monostearate, glyceryl stearate, polyglyceryl-4-oleate, sorbitan acylate, sucrose acylate, PEG-150 laurate, PEG-400 monolaurate, polyoxyethylene monolaurate, polysorbates, polyoxyethylene octylphenylether, PEG-1000 cetyl ether, polyoxyethylene tridecyl ether, polypropylene glycol butyl ether, Poloxamer® 401, stearoyl monoisopropanolamide, and polyoxyethylene hydrogenated tallow amide. Examples of amphoteric surfactants include sodium N-dodecyl-.beta.-alanine, sodium N-lauryl-.beta.-iminodipropionate, myristoamphoacetate, lauryl betaine and lauryl sulfobetaine.

### C. Chemotherapeutic Agents

The loading of the active agent(s) typically is from about 1 to about 90 wt %, preferably from about 10 to about 70 wt %, more preferably from about 30 to about 50%.

Representative chemotherapeutic agents include cisplatin, BCNU, taxol and other taxanes, doxorubicin, anti-estrogens or anti-estrogen receptors such as tamoxifen and fulvestrant. Other agents include hormonal agents or other biological agents to prevent or delay disease recurrence.

### D. Imaging Agents

Suitable diagnostic agents include any variety of medical imaging and diagnostic agents including, for example, MRI-based imaging such as iron oxide particles (including, for example superparamagnetic iron oxide, or SPIO, particles) and gadolinium-containing agents, among others. The particle compositions can also be prepared containing any of a variety of other dyes, contrast agents, fluorescent markers, imaging agents, and radiologic agents used in any variety of medical diagnostic and imaging technologies.

### E. Excipients, Carriers, and Additives

The particle composition may further contain one or more pharmaceutically acceptable excipients, carriers, and additives. As used herein, the “carrier” is all components present in the pharmaceutical formulation other than the active ingredient or ingredients. The term “carrier” includes, but is not limited to, solvents, suspending agents, dispersants, buffers, pH modifying agents, isotonicity modifying agents, preservatives, antimicrobial agents, and combinations thereof.

In addition to a therapeutic or diagnostic agent (or possibly other desired molecules for delivery), the particles can include excipients such as a sugar, such as lactose, a protein, such as albumin, and/or a surfactant. If the agent to be delivered is negatively charged, protamine or other positively charged molecules can be added to provide a lipophilic complex which results in the sustained release of the negatively charged agent. Negatively charged molecules can be used to render insoluble positively charged agents. Additionally, the coating material may contain conventional carriers such as plasticizers, pigments, colorants, glidants, stabilization agents, pore formers and surfactants.

Other additives include those useful for processing or preparation of the particle composition, can aid in the incorporation or stability of the active agent, or can be useful in modifying performance of the particle composition, including, for example, modifying the rate of drug release, drug stability, water uptake, polymer degradation, among others.

The one or more excipients can be incorporated during preparation of the particles. The excipients can be added separately into the polymer solution itself, can be incorporated into a first solution containing the active agent dissolved or dispersed into a first solvent, or can be added into the polymer solution before, during, or after the active agent is added into the polymer solution.

Delayed release dosage units can be prepared, for example, by coating a drug or a drug-particle composition with a coating material, for example, bioerodible, gradually hydrolyzable, gradually water-soluble, and/or enzymatically degradable polymers, or conventional “enteric” polymers. Enteric polymers become soluble in the higher pH environment of the lower gastrointestinal tract or slowly erode as the dosage form passes through the gastrointestinal tract, while enzymatically degradable polymers are degraded by bacterial enzymes present in the lower gastrointestinal tract, particularly in the colon, but may also be used in other tissues which also contain enzymes that will biodegrade the coating material. Suitable coating materials for effecting delayed release include, but are not limited to, cellulosic polymers such as hydroxypropyl cellulose, hydroxyethyl cellulose, hydroxymethyl cellulose, hydroxypropyl methyl cellulose, hydroxypropyl methyl cellulose acetate succinate, hydroxypropylmethyl cellulose phthalate, methylcellulose, ethyl cellulose, cellulose acetate, cellulose acetate phthalate, cellulose acetate trimellitate and carboxymethylcellulose sodium; acrylic acid polymers and copolymers, preferably formed from acrylic acid, methacrylic acid, methyl acrylate, ethyl acrylate, methyl methacrylate and/or ethyl methacrylate, and other methacrylic resins that are commercially available under the tradename Eudragit®. (Rohm Pharma; Westerstadt, Germany), including Eudragit®. L30D-55 and L100-55 (soluble at pH 5.5 and above), Eudragit®. L-100 (soluble at pH 6.0 and above), Eudragit®. S (soluble at pH 7.0 and above, as a result of a higher degree of esterification), and Eudragits®. NE, RL and RS (water-insoluble polymers having different degrees of permeability and expandability); vinyl polymers and copolymers such as polyvinyl pyrrolidone, vinyl acetate, vinylacetate phthalate, vinylacetate crotonic acid copolymer, and ethylene-vinyl acetate copolymer; enzymatically degradable polymers such as azo polymers, pectin, chitosan, amylose and guar gum; zein and shellac. Combinations of different coating materials may also be used. Multi-layer coatings using different polymers may also be applied.

Examples of suitable coating materials include, but are not limited to, cellulose polymers such as cellulose acetate phthalate, hydroxypropyl cellulose, hydroxypropyl methylcellulose, hydroxypropyl methylcellulose phthalate and hydroxypropyl methylcellulose acetate succinate; polyvinyl acetate phthalate, acrylic acid polymers and copolymers, and methacrylic resins that are commercially available under the trade name Eudragit® (Roth Pharma, Westerstadt, Germany), Zein, shellac, and polysaccharides.

If the molecules are hydrophilic and tend to solubilize readily in an aqueous environment, another method for achieving sustained release is to use cholesterol or very high surfactant concentration. This complexation methodology also applies to particles that are not aerodynamically light.

### II. Methods of Making Microparticle Compositions

The following are representative methods for forming microparticles.

Spray Drying

In spray drying, the core material to be encapsulated is dispersed or dissolved in a solution. Typically, the solution is aqueous and preferably the solution includes a polymer. The solution or dispersion is pumped through a micronizing nozzle driven by a flow of compressed gas, and the resulting aerosol is suspended in a heated cyclone of air, allowing the solvent to evaporate from the microdroplets. The solidified microparticles pass into a second chamber and are trapped in a collection flask.

Interfacial Polycondensation

Interfacial polycondensation is used to microencapsulate a core material in the following manner. One monomer and the core material are dissolved in a solvent. A second monomer is dissolved in a second solvent (typically aqueous) which is immiscible with the first. An emulsion is formed by suspending the first solution through stirring in the second solution. Once the emulsion is stabilized, an initiator is added to the aqueous phase causing interfacial polymerization at the interface of each droplet of emulsion.

Hot Melt Encapsulation

In hot melt microencapsulation, the core material (to be encapsulated) is added to molten polymer. This mixture is suspended as molten droplets in a nonsolvent for the polymer (often oil-based) which has been heated to approximately 10° C. above the melting point of the polymer. The emulsion is maintained through vigorous stirring while the nonsolvent bath is quickly cooled below the glass transition of the polymer, causing the molten droplets to solidify and entrap the core material.

Solvent Evaporation Microencapsulation

In solvent evaporation microencapsulation, the polymer is typically dissolved in a water immiscible organic solvent and the material to be encapsulated is added to the polymer solution as a suspension or solution in an organic solvent. An emulsion is formed by adding this suspension or solution to a beaker of vigorously stirring water (often containing a surface active agent, for example, polyethylene glycol or polyvinyl alcohol, to stabilize the emulsion). The organic solvent is evaporated while continuing to stir. Evaporation results in precipitation of the polymer, forming solid microcapsules containing core material.

The solvent evaporation process can be used to entrap a liquid core material in a polymer such as PLA, PLA/PGA copolymer, or PLA/PCL copolymer microcapsules. The polymer or copolymer is dissolved in a miscible mixture of solvent and nonsolvent, at a nonsolvent concentration which is immediately below the concentration which would produce phase separation (i.e., cloud point). The liquid core material is added to the solution while agitating to form an emulsion and disperse the material as droplets. Solvent and nonsolvent are vaporized, with the solvent being vaporized at a faster rate, causing the polymer or copolymer to phase separate and migrate towards the surface of the core material droplets. This phase-separated solution is then transferred into an agitated volume of nonsolvent, causing any remaining dissolved polymer or copolymer to precipitate and extracting any residual solvent from the formed membrane. The result is a microcapsule composed of polymer or copolymer shell with a core of liquid material.

Solvent evaporation microencapsulation can result in the stabilization of insoluble drug particles in a polymeric solution for a period of time ranging from 0.5 hours to several months. The stabilization of insoluble drug particles within the polymeric solution could be critical during scale-up. By stabilizing suspended drug particles within the dispersed phase, said particles can remain homogeneously dispersed throughout the polymeric solution as well as the resulting polymer matrix that forms during the process of microencapsulation. The homogeneous distribution of drug particles can be achieved in any kind of device, including microparticles, nanoparticles, rods, films, and other device.

Solvent evaporation microencapsulation (SEM) has several advantages. SEM allows for the determination of the best polymer-solvent-insoluble particle mixture that will aid in the formation of a homogeneous suspension that can be used to encapsulate the particles. SEM stabilizes the insoluble particles or within the polymeric solution, which will help during scale-up because one will be able to let suspensions of insoluble particles sit for long periods of time, making the process less time-dependent and less labor intensive. SEM allows for the encapsulated particles to remain suspended within a polymeric solution for up to 30 days, which may increase the amount of insoluble material entrapped within the polymeric matrix, potentially improving the physical properties of the drug delivery vehicle. SEM allows for the creation of microparticles or nanoparticles that have a more optimized release of the encapsulated material. For example, if the insoluble particle is localized to the surface of the microparticle or nanoparticle, the system will have a large ‘burst’ effect. In contrast, creating a homogeneous dispersion of the insoluble particle within the polymeric matrix will help to create a system with release kinetics that begin to approach the classical ‘zero-ordered’ release kinetics that are often perceived as being ideal in the field of drug delivery).

In one embodiment, the microparticles are prepared using an emulsion-based methodology. Examples include emulsion-solvent extraction methods (for example, U.S. Pat. Nos. 5,407,609; 5,650,173; 6,537,586; 6,540,393; 5,654,008), emulsion-solvent evaporation methods (for example, U.S. Pat. No. 4,530,840), or combinations of extraction and evaporation techniques (for example, U.S. Pat. No. 6,440,493). In these methods of preparing microparticle compositions, a polymer solution is typically prepared by dissolving the polymer or admixture of two or more polymers in a suitable solvent. The solvent can be a single solvent or a cosolvent. Generally speaking, a single solvent or an admixture of two or more solvents is referred to as a “solvent system.”

The active agent is typically added to the polymer solution, either as a solid or as a solution or suspension. The active agent may or may not be soluble in the polymer solution. In some embodiments, the active agent can be added after first dissolving or suspending the active agent in a solvent system (the “first solvent”) then adding this solution or suspension into the polymer solution. The active agent can be dissolved in the first solvent and, upon adding this solution to the polymer solution, the active agent can remain dissolved in the resulting polymer solution. Alternatively, the addition of the solution containing the active agent to the polymer solution can result in the active agent precipitating out of solution to a greater or lesser extent, depending on the overall solubility of the active agent in the resulting solution.

The first solvent (i.e., the solvent system used to dissolve or suspend the active agent) can be fully soluble in the polymer solution. In another aspect, the first solvent can be only partially soluble (or miscible) in the resulting polymer solution and a liquid-liquid emulsion is formed. In still another aspect, the first solvent can be only slightly soluble in the polymer solution; alternatively, the solvent can be nearly or virtually insoluble in the polymer solution. In situations when the first solvent is not fully soluble in the polymer solution, then a liquid-liquid emulsion will form. This emulsion can be either an oil-in-water emulsion or a water-in-oil emulsion depending on the particular solvent systems used to prepare the polymer and drug solutions. Preparing polymer solutions in the form of an emulsion is not uncommon and is often described as the “double-emulsion” technique for preparing microparticle compositions.

The active agent can be distributed homogeneously through out the microparticle. Alternatively, the active agent can be distributed heterogeneously in the microparticle matrix, i.e. encapsulated within (e.g., in the interior) of the microparticle or the exterior regions of the microparticle.

Solvent Removal Microencapsulation

In solvent removal microencapsulation, the polymer is typically dissolved in an oil miscible organic solvent and the active agent to be encapsulated is added to the polymer solution as a suspension, dissolved in water, or as a solution in and organic solvent. Surface active agents can be added to improve the dispersion of the material to be encapsulated. An emulsion is formed by adding this suspension or solution to an oil with stirring, in which the oil is a nonsolvent for the polymer and the polymer/solvent solution is immiscible in the oil. The organic solvent is removed by diffusion into the oil phase while continuing to stir. Solvent removal results in precipitation of the polymer, forming solid microcapsules containing core material.

Phase Separation Microencapsulation

In phase separation microencapsulation, the material to be encapsulated is dispersed in a polymer solution with stirring. While continually stirring to uniformly suspend the material, a nonsolvent for the polymer is slowly added to the solution to decrease the polymer's solubility. Depending on the solubility of the polymer in the solvent and nonsolvent, the polymer either precipitates or phase separates into a polymer rich and a polymer poor phase. Under proper conditions, the polymer in the polymer rich phase will migrate to the interface with the continuous phase, encapsulating the core material in a droplet with an outer polymer shell.

Spontaneous Emulsification

Spontaneous emulsification involves solidifying emulsified liquid polymer droplets by changing temperature, evaporating solvent, or adding chemical cross-linking agents. The physical and chemical properties of the encapsulant, and the material to be encapsulated, dictates the suitable methods of encapsulation. Factors such as hydrophobicity, molecular weight, chemical stability, and thermal stability affect encapsulation.

Coacervation

Encapsulation procedures for various substances using coacervation techniques have been described in the prior art, for example, in GB-B-929 406; GB-B-929 401; U.S. Pat. Nos. 3,266,987; 4,794,000 and 4,460,563. Coacervation is a process involving separation of colloidal solutions into two or more immiscible liquid layers (Ref. Dowben, R. General Physiology, Harper & Row, New York, 1969, pp. 142-143.). Through the process of coacervation compositions comprised of two or more phases and known as coacervates may be produced. The ingredients that comprise the two phase coacervate system are present in both phases; however, the colloid rich phase has a greater concentration of the components than the colloid poor phase.

Phase Inversion Nanoencapsulation (“PIN”)

In PIN, a polymer is dissolved in an effective amount of a solvent. The agent to be encapsulated is also dissolved or dispersed in the effective amount of the solvent. The polymer, the agent and the solvent together form a mixture having a continuous phase, wherein the solvent is the continuous phase. The mixture is introduced into an effective amount of a nonsolvent to cause the spontaneous formation of the microencapsulated product, wherein the solvent and the nonsolvent are miscible. PIN has been described by Mathiowitz et al, in U.S. Pat. Nos. 6,131,211 and 6,235,224. A hydrophobic agent is dissolved in an effective amount of a first solvent that is free of polymer. The hydrophobic agent and the solvent form a mixture having a continuous phase. A second solvent and then an aqueous solution are introduced into the mixture. The introduction of the aqueous solution causes precipitation of the hydrophobic agent and produces a composition of micronized hydrophobic agent having an average particle size of 1 micron or less.

Melt-Solvent Evaporation Method

In the melt-solvent evaporation method, the polymer is heated to a point of sufficient fluidity to allow ease of manipulation (for example, stirring with a spatula). The temperature required to do this is dependent on the intrinsic properties of the polymer. For example, for crystalline polymers, the temperature will be above the melting point of the polymer. After reaching the desired temperature, the agent is added to the molten polymer and physically mixed while maintaining the temperature. The molten polymer and agent are mixed until the mixture reaches the maximum level of homogeneity for that particular system. The mixture is allowed to cool to room temperature and harden. This may result in melting of the agent in the polymer and/or dispersion of the agent in the polymer. This can result in an increase in solubility of the drug when the mixture is dissolved in organic solvent. The process is easy to scale up since it occurs prior to encapsulation. High shear turbines may be used to stir the dispersion, complemented by gradual addition of the agent into the polymer solution until the desired high loading is achieved. Alternatively the density of the polymer solution may be adjusted to prevent agent settling during stirring.

This method increases microparticle loading as well as uniformity of the resulting microparticles and of the agent within the microparticles. When an agent is formed into microspheres by double-emulsion solvent evaporation, transfer of the agent from the inner phase to the outer water phase can be prevented. This makes it possible to increase the percentage of agent entrapped within the microspheres, resulting in an increased amount of the drug in the microspheres.

The distribution of the agent in particles can also be made more uniform. This can improve the release kinetics of the agent. Generally, the agent is dissolved or dispersed together with a substance that has a high molecular weight in an organic solvent composition; with or without non-ionic surfactants of various hydrophilic-lipophilic ratios. The composition is introduced into an aqueous solution that contains a surfactant like PVA. The water-insoluble solvent forms an oil phase (inner phase) and is stirred into the aqueous solution as a water phase (outer phase). The O/W emulsion is combined with fresh water that contains PVA and is stirred to help aid the solvent evaporation. The aqueous solution contains an activator such as polyvinyl alcohol, whereby the oil phase is enclosed as small droplets within the aqueous solution as shells.

The microparticle can also be in the form of a multilayer microparticle. Multilayer microparticles can be prepared using any multiple steps or combination of steps of the techniques described above. This can include the preparation of a “core” particle by methods known in the art (including, for example, extrusion-spheronization or other granulation processes) that are then further processed in order to add one or more polymer layers to the core. For example, a first particle (a “core” particle) can be prepared by an emulsion-based method and treated using a second process such as a fluid-bed process in order to incorporate a second layer of material on the outside of the core particle. Alternatively, the multilayer microparticles can be prepared using processes that produce multiple layer particles directly (i.e., without the need for additional steps). Exemplary processes includes multiple or dual nozzle (or jet) configurations (otherwise termed nozzle-in-nozzle or dual nozzle configurations) for the preparation of multi-layer particles (for example, U.S. Pat. No. 6,669,961).

The polymer used to prepare the microparticle compositions can be a single polymer or can be a mixture of two or more polymers that are admixed together before preparing the microparticles. In one aspect, preparing the admixture of polymers can simply involve weighing out the appropriate quantities of the selected polymers and then using these individual materials to prepare a solution that is then used to prepare the microparticle composition. In another aspect, polymer solutions can be prepared separately using different polymers; then these polymer solutions can be combined prior to making the microparticle composition. In another aspect, the admixture of polymers can involve first combining and blending the various individual polymers in order to make a blend of the dry polymer solids; then that blend can be used to prepare the polymer solution that is used in the making of the microparticle composition. In still another aspect, the polymer admixture can be prepared by dissolving or dispersing the individual polymers in a solvent which is then evaporated leaving behind an admixture from a solvent-casting and evaporation process. In another aspect, the polymer admixture can be prepared by dissolving the individual polymers in a suitable solvent and then freezing and lyophilizing the sample to remove the solvent. In still another aspect, the polymer admixture can be prepared from the various individual polymers by supercritical fluid techniques.

The admixture of polymers can be prepared in the absence of a active agent. In another aspect, the polymer admixture can be prepared with some or the entire amount of the active agent that is used in the preparation of the microparticle composition.

Spray Drying, Atomizing and Fluid-Bed Methods

The microparticle compositions described herein can be prepared by a variety of methods known in the art including spray-drying; fluid-bed techniques; techniques that utilize spraying of solutions through nozzles (or jets) either into air or into liquids in order to prepare microparticles; cryogenic spray techniques (See U.S. Pat. No. 5,989,463, for example); ultrasonic spraying through nozzles (or jets) without or with the presence of applied electrical potential (e.g., electrostatic spraying) as described in U.S. Pat. No. 6,669,961; supercritical fluid techniques for the preparation of microparticle compositions; or any of the general techniques involving polymer precipitation or phase separation or coacervation and any combinations therein.

Methods for Determining Particle Size

The mean particle diameter is the average diameter over the whole distribution. There are different types of means that can be calculated from the same set of particle size distribution data. For example, if there are ten spheres of diameters ranging from 1 to 10, the sum of their diameters from the distribution can be represented by ten spheres each of diameter 5.5; but the sum of their volumes has to be represented by ten spheres each having a diameter of 6.72. Each particle characterization technology will “see” the same sample differently. In the language of statistics, different technologies see particles through different “weighting factors.” For example, when using tunneling electron microscopy (TEM), particles are measured on the basis of their number, but when using laser diffraction, the light scattering intensity of particles is detected on the basis of their volume. Different mean values thus have to be defined and used.

The microparticle compositions include particles having a diameter from about 10 nm to about 1000 microns. In general, the microparticle compositions can be prepared within this size range that are of a suitable size, or range of sizes, for use in any variety of medical, surgical, clinical, cosmetic, medical device, pharmaceutical and/or drug-delivery applications. In one aspect, the microparticles have a size in the range of from about 250 to about 1000 microns. In another aspect, the microparticles have a size in the range of from about 100 to about 250 microns. In another aspect, as in the case of microparticle compositions typically used for subcutaneous (SC) or intramuscular (IM) administration, the microparticles have a diameter in the range from about 20 to about 150 microns. In some embodiment, the microparticles have a diameter in the range from about 20 to about 50 microns, preferably from about 20 to about 40 microns. In other embodiment, the microparticles have a diameter in the range from about 1 to about 30 microns, preferably from about 1 to about 20 microns, more preferably from about 1 to about 10 microns.

Modification of Polymers or Particles

The polymers can be modified by increasing the number of carboxylic groups accessible during biodegradation, or on the polymer surface to alter bioadhesion or to attach ligands that may be useful for targeting of the microparticles to specific cell markers. The polymers can also be modified by binding amino groups to the polymer. The polymers can also be modified using any of a number of different coupling chemistries that covalently attach drugs or other molecules to the polymeric microspheres.

One protocol involves the “activation” of hydroxyl groups on polymer chains with the agent, carbonyldiimidazole (CDI) in aprotic solvents such as DMSO, acetone, or THF. CDI forms an imidazolyl carbamate complex with the hydroxyl group which may be displaced by binding the free amino group of a ligand such as a protein. The reaction is an N-nucleophilic substitution and results in a stable N-alkylcarbamate linkage of the ligand to the polymer. The “coupling” of the ligand to the “activated” polymer matrix is maximal in the pH range of 9-10 and normally requires at least 24 hrs. The resulting ligand-polymer complex is stable and resists hydrolysis for extended periods of time.

Another coupling method involves the use of 1-ethyl-3-(3-dimethylaminopropyl) carbodiimide (EDAC) or “water-soluble CDI” in conjunction with N-hydroxylsulfosuccinimide (sulfa NHS) to couple the exposed carboxylic groups of polymers to the free amino groups of ligands in a totally aqueous environment at the physiological pH of 7.0. Briefly, EDAC and sulfo-NHS form an activated ester with the carboxylic acid groups of the polymer which react with the amine end of a ligand to form a peptide bond. The resulting peptide bond is resistant to hydrolysis. The use of sulfo-NHS in the reaction increases the efficiency of the EDAC coupling by a factor of ten-fold and provides for exceptionally gentle conditions that ensure the viability of the ligand-polymer complex.

Either of these protocols can be used to “activate” almost all polymers containing either hydroxyl or carboxyl groups in a suitable solvent system that will not dissolve the polymer matrix.

A useful coupling procedure for attaching ligands with free hydroxyl and carboxyl groups to polymers involves the use of the cross-linking agent, divinylsulfone. This method is useful for attaching sugars or other hydroxylic compounds with bioadhesive properties to hydroxylic matrices. Briefly, the activation involves the reaction of divinylsulfone to the hydroxyl groups of the polymer, forming the vinylsulfonyl ethyl ether of the polymer. The vinyl groups will couple to alcohols, phenols and even amines. Activation and coupling take place at pH 11. The linkage is stable in the pH range from 1-8 and is suitable for transit through the intestine. Any suitable coupling method known to those skilled in the art for the coupling of ligands and polymers with double bonds, including the use of UV crosslinking, may be used for attachment of drugs or other molecules to the polymeric microspheres described herein.

A. Preparation of Suspensions for Injection

For injection, it is preferable to suspend the microparticle composition in a liquid suspending medium, which is also called an injection vehicle or fluid or diluent prior to administration. These suspensions are typically heterogeneous systems containing the solid, essentially insoluble dispersed material (the microparticle composition) suspended or disbursed in a liquid phase (the injection vehicle). The injection vehicle is typically sterile, stable, and capable of being delivered through a small-diameter needle without clogging or otherwise blocking the delivery of the microparticle suspension.

The injection vehicle or liquid phase can be fan aqueous or non-aqueous pharmaceutically acceptable liquid. The injection vehicle can be of relatively low or high viscosity but should be of sufficient viscosity so that the resulting suspension formed from the microparticle composition is of suitable viscosity to be passed through the desired needle. The vehicles typically are sterile water, saline, or phosphate buffered saline. Suitable nonaqueous injection vehicles include, but are not limited to, fluorinated liquid vehicles such as polyfluoroalkylmethylsiloxanes, Miglyol or other pharmaceutically acceptable oils and oil-based vehicles.

The injection vehicle may contain one or more viscosity-modifying agents and/or surfactants. Other suitable additives include, but are not limited to, buffers, osmotic agents, and preservatives. Examples of viscosity-modifying agents include synthetic polymers such as poloxamers, Pluronics, or polyvinyl pyrrolidone; polysaccharides, such as sodium carboxymethyl cellulose (CMC); and natural polymers, such as gelatin, hyaluronic acid, or collagene.

The microparticle compositions are typically dispersed or suspended in the injection vehicle. The concentration of microparticles dispersed or suspended in a particular volume of injection vehicle can range from dilute to concentrated. As used herein, the concentration of the microparticles refers to the solids loading of the microparticles in the liquid injection vehicle. The required concentration of solids in the suspension can be determined by the application or by the strength or activity of the active agent or both. In one embodiment, the concentration of solids in the suspension is from about 0.1 wt % to about 75 wt %. Preferred solids contents include from about 0.1 wt % to about 1 wt %, from about 1 wt % to about 10 wt %, from about 5 wt % to about 50 wt %, or from about 50 wt % to about 75 wt %.

B. Sterilization

The microparticle compositions can be prepared aseptically and used without any further processing in order to have a sterile product suitable for administration to a subject. In other aspects, the microparticle composition can be prepared non-aseptically and then be subjected to a terminal sterilization operation in order to have a sterile product that is suitable for administration to a subject. Without intending to be limiting, terminal sterilization operations can include exposure to irradiation such as gamma-irradiation or electron-beam radiation, or exposure to ethylene oxide gas.

### III. Methods of Administration

Particles providing prolonged release of chemotherapy are injected or implanted into surgical sites in the breast following removal of cancerous tissue from a patient. In one embodiment, the particles are designed to not release formulation for approximately two to three weeks after surgery so as to not inhibit healing; in another embodiment particles are not administered until two to three weeks after surgery, and release immediately. The particles then release an effective amount of a chemotherapeutic such as a taxane to inhibit proliferation of any remaining cancer cells at or near the surgical site. This may also help prevent overproliferation leading to scarring with the surgical region. In a preferred embodiment, release occurs for six to twelve months. Upon a negative re-examination, the particles are re-injected into the surgical site to prevent reoccurrence. In a particularly preferred embodiment the particles also contain an imaging agent so that they can be imaged and/or removed if necessary.

The microparticles can be administered as a powder, directly at or around the site after surgery, or injected later during a follow up procedure. The microparticle compositions described herein are generally administered by injection, for example a 16 to 31 gauge needle, depending on the application. The compositions can also be administered through a larger diameter tube, catheter, trocar, infusion tubing, or endoscopy/arthroscopic tubes.

Agents will be administered either by palpation of the lumpectomy site, with image guidance using either mammographic, stereotactic, sonographic guidance or with MRI or other imaging modalities for guidance of the agent into or adjacent to the site of cancer therapy. Additionally, the agent may be administered via the breast ducts to prevent or treat cancer. Agents will be administered prior to the development of breast cancer or at the time of initial therapy and/or at intervals as required based on the degradation of the active agent or the carrier agent. Agent will be administered to either treat residual cancer or decrease the likelihood of recurrence at the time of initial treatment and/or at intervals to decrease the recurrence of cancer. Additionally, agent will be administered to decrease, delay or prevent fibrotic reaction from therapy, either surgical, radiation or chemotherapy. Treatment will continue at interval throughout life to decrease the risk of cancer, to prevent cancer or to treat the cosmetic result of treatment for or prophylaxis of breast cancer.

The patient may be female or male. Typically the patient is a woman. While breast cancer is rare in men, it has similar treatments to breast cancer found in women. For example, treatment for male breast cancer usually involves surgery, such as a lumpectomy or a mastectomy. Other treatments include radiation, chemotherapy and/or hormone therapy. The particles described herein may be administered as described above to decrease the risk of cancer, to prevent cancer or to treat the cosmetic result of treatment for or prophylaxis of breast cancer in a male or female patient in need thereof.

We claim:

1. A method for decreasing the reoccurrence of breast cancer in a patient who has been treated for breast cancer comprising administering at or near a site where breast cancer cells were removed particles releasing an effective amount of a chemotherapeutic to inhibit proliferation of any remaining cancer cells at or near the surgical site.

2. The method of claim 1 wherein the particles release an effective amount of chemotherapeutic for a period of between six and twelve months.

3. The method of claim 1 wherein the particles are administered at least ten days after surgery to remove the cancer cells.

4. The method of claim 1 wherein the particles are administered following a negative diagnostic scan after surgery to remove the cancer cells.

5. The method of claim 3 wherein the particles release an effective amount of chemotherapeutic to inhibit scarring or formation of fibrotic tissue.

6. The method of claim 1 wherein the particles further comprising an imaging agent.

7. The method of claim 6 wherein the particles are imaged after administration.

8. The method of claim 1 wherein the chemotherapeutic is selected from the group consisting of cisplatin, BCNU, taxol and other taxanes, doxorubicin, anti-estrogens and anti-estrogen receptors.

9. The method of claim 1, wherein the patient is a woman.

10. A microparticulate formulation for use in the method of claim 1.
